(19) United States
(12) Patent Application Publication
(10) Pub. No.: US 2026/0222524 A1
(43) Pub. Date: Jul. 30, 2026

(54) A SYSTEM FOR SELECTIVELY PROJECTING AN OBJECT OF INTEREST ONTO A PREDEFINED RETINAL AREA

(71) Applicant: La Science SAS, Paris (FR)

(72) Inventor: Jacques Feldmar, Saint Mande (FR)

(73) Assignee: La Science SAS, Paris (FR)

(21) Appl. No.: 19/143,639

(22) PCT Filed: Jan. 30, 2024
(86) PCT No.: PCT/EP2024/052245

§ 371 Date: Jun. 26, 2025

Related U.S. Application Data
(63) Continuation of application No. PCT/EP2023/052180, filed on Jan. 30, 2023.

(51) Int. Cl. H04N 9/31; A61F 9/08; H04N 23/611; H04N 23/62; H04N 23/69

(52) U.S. Cl. CPC H04N 9/3194; A61F 9/08; H04N 9/3152; H04N 9/3185; H04N 23/611; H04N 23/62; H04N 23/69; H04N 9/3182

(57) ABSTRACT

The invention relates to a system for projecting an object of interest into a human eye in a selective manner. In particular, the invention relates to a system for supporting the detection and selection of an object of interest and to project said object of interest onto a predefined retinal area of the human eye. Accordingly, a system (10) for projecting at least a portion of an object of interest (28) onto a predefined retinal area (22) of a human eye of a user is suggested, comprising a camera (12) for capturing an image, a projector device (14) for projecting a light beam (20) into a human eye based on an inputted signal, at least a first user input device (26), and a processor (16) being in communication with the camera (12), at least the first user input device (26), and the projector device (14). According to the invention, the processor (16) is further configured to detect at least a portion of the object of interest (28) in a captured image based on a user input received from the first user input device (26) and to output a signal corresponding to a portion of the captured image to the projector device (14) and to output a signal for centering at least said portion of the object of interest (28) relative to the predefined retinal area (22).

### TECHNICAL FIELD

The invention relates to a system for projecting an object of interest onto the retinal area of a human eye in a selective manner. In particular, the invention relates to a system for supporting the detection and selection of an object of interest and to project said object of interest onto a predefined retinal area of the human eye.

### TECHNOLOGICAL BACKGROUND

Retinal malfunction, particularly caused by degenerative retinal diseases, is a leading reason for visual impairment or even blindness.

For at least partially restoring a patient's visual function, it is known to modify a retinal area by making use of a retinal implant or, in other words, a retinal prosthesis. In this regard, several different types of retinal implants are known, which are based on different working principles. Retinal implants have in common that they are usually positioned suprachoroidally, epiretinally or subretinally in the eye of the patient, such that they can replace the function of the damaged photoreceptor cells. In this regard, information about a visual scene is captured with a camera and then transmitted to an electrode array implanted in the retina.

Among common retinal implants, implants are known which comprise skin-penetrating wires. These wires introduce risks of infection and scarring. Thus, more modern implants use different wireless techniques, for instance by delivering power and visual information through inductive coils. Furthermore, it is known to deliver power inductively and visual information optically through the pupil of the eye, or to deliver both visual information and power optically.

A particularly beneficial type of wireless information transfer retinal implants is based on projecting stimulation patterns of light, e.g. infrared light, into the eye. When the gaze direction is such that some part of the implant is illuminated by part of the stimulation pattern, the implant converts that part of the signal to electrical current that stimulates the retina cells accordingly. The retinal implant is typically an array composed of stimulation electrodes or pixels. Each pixel has one or several photodiodes that capture the light delivered from a visual processor and converts it into electrical current for stimulation.

More than one implant array can be implanted, e.g. placed in the subretinal space, typically in or close to the foveal area.

An alternative approach is the use of optogenetic applications to modify or improve a targeted retinal area in the eye, such as reactivating the photosensibility of the targeted area of the retina (i.e. reactivated retinal area). “Optogenetics” refers to the combination of genetics and optics to control well-defined events within specific cells of living tissue. “Optogenetics” encompasses in (i) genetically modifying target cells in order to render them sensitive to light by the expression of exogenous photoreactive proteins in cellular membrane and (ii) provision of an illuminating optical device projecting stimulation patterns of light able to stimulate said photoreactive proteins.

The projecting of light or a light beam may be provided within a particular wavelength range having a specific irradiance adapted for the activation of the retinal implants or the exogenous photoreactive proteins in the cellular membrane. Accordingly, the light beam may be output e.g. in the infrared range, thereby ensuring that areas of the retina that have not been modified are not affected by the projected light.

Furthermore, for projecting light or a light beam into a human eye, it is known to use a projector device that projects the light beam onto the human eye, wherein the light beam may be adapted to the size of the pupil of the human eye. That approach may advantageously ensure that essentially the entire light beam is projected onto the retina of the human eye and that the information received from a camera input and converted into the light is essentially processed. From U.S. Pat. No. 11,343,420 B1 a system for an eye-based external camera selection and control is known, wherein images of an external camera are projected to a human eye based on a visual input and visual selection of a user. Accordingly, the user visually focuses on a particular visually perceivable area, thereby triggering the activation of a camera and synchronization of said camera with a projector integrated in a contact lens of the user. The user visually identifies the projected image and any objects contained therein. US 2022/066549 A1 also discloses the use of a projector being integrated in a contact lens of the user. It also discloses that objects within the field of view may be visually selected by means of a corresponding position of the user's eye, wherein corresponding information regarding the object is projected into the user's eye.

US 2022/001134 A discloses systems for assisting in augmented reality, wherein artificial content is selected by a user and rendered in images corresponding to the field of view of the user. The combined field of view with the artificial content is hence projected to a user having full vision. Based on gestures of the user the artificial content, e.g. its relative position, may be modified within the rendered and projected image.

### SUMMARY OF THE INVENTION

Starting from the known prior art there is a need to further facilitate that an object of interest may be perceived by a patient in a convenient manner.

According to the invention, it has been recognized that even with the provision of a proper light beam size to enter the pupil of the human eye it may become difficult for a patient to obtain the desired visual information. That finding is due to the fact that a field of view of a camera is typically larger than a field of view of the corresponding light beam, such that the light beam cannot provide the entirety of information available within a captured image. To facilitate that a patient may focus on an object of interest, the light beam and the camera are typically aligned in a predefined manner, such that a movement of the camera results in a corresponding change of information perceived by the patient.

However, the size of a retinal implant or reactivated retinal area of a human eye is typically smaller than the size of the light beam, such that the object of interest may not or only be partially perceived by the patient. That effect may lead to insecurity or confusion at the patient end, in particular, since the resolution of a retinal implant or capability of a reactivated retinal area to transmit signals with appropriate optical resolution and number of grey levels is generally low. If the portion of the light beam projecting onto the modified retina (i.e. a retinal implant or reactivated retinal area) hence does not allow identification of the object of interest, the patient may be confused or even be dazzled, e.g. in case of high contrasts or intensities captured in the image and provided by the projected light beam.

With an increasing number of objects or increasing amount of visual information being present, it will be more difficult for the patient to identify an object of interest. Even if the gaze of the patient or the center of the camera is pointed towards such object, a so-called crowding effect may render it difficult for a patient to distinguish the object of interest from one or more directly adjacent objects of interests. By the same token, if a magnification or zoom factor of the camera is not properly set, the provided resolution may be insufficient for the patient to identify the object of interest or a portion thereof.

It is hence an object of the present invention to provide an improved system for projecting an object of interest, or a portion thereof, onto a retinal area of a human eye, in particular to facilitate a centering of the system and/or predefined retinal area with an object of interest or a portion thereof. This improved system may further facilitate zoom level adjustment of the system and/or predefined retinal area with an object of interest or a portion thereof.

The above object is solved by means of a system for projecting an optical pattern of interest onto a predefined retinal area of a human eye comprising the features of claim 1. Further preferred embodiments are presented in the dependent claims, the description and the Figures.

Accordingly, in one aspect, a system for projecting at least a portion of an object of interest onto a predefined retinal area of a human eye of a user is suggested, comprising a camera for capturing an image, a projector device for projecting a light beam onto the fundus of a human eye based on an inputted signal, at least a first user input device, and a processor being in communication with the camera, with at least the first user input device, and with the projector device. According to the invention, the processor is configured to detect at least a portion of the object of interest in a captured image based on a user input received from the first user input device and to output a signal corresponding to a portion of the captured image to the projector device and to output a signal for centering at least said portion of the object of interest relative to the predefined retinal area and preferably for adjusting a zoom level.

The present inventors have identified that the patient, even in the case of a partial loss of central vision and/or peripheral vision, may typically use his remaining vision capabilities to align the camera with an object of interest. For example, the patient may still use the peripheral vision in case of full or partial loss of central vision, and, vice versa, may use at least parts of remaining central vision, if sufficient for aligning the camera.

However, if a larger number of objects are present and/or if a potential object of interest is present at a position that is not readily perceivable, it may be difficult for a patient to establish whether the object of interest is present at all. For example, if a larger number of objects are present in vicinity of each other, the object of interest may not be readily identifiable by the patient. Identification of the object of interest may become even more difficult, if such objects are positioned further away from the patient and/or are positioned at varying distances. Under such circumstances, the combination of a crowding effect and an improper magnification, i.e. a loss of resolution, may result in a presentation of confusing information to the patient to the extent that the patient is not capable of identifying the object of interest. This applies both to central vision and peripheral vision.

By providing the user input, the processor may detect the presence of such object of interest in the captured image in a semi-automated manner. In this regard, the object of interest may be particularly understood as a physical and/or graphical object that is actually present in front of the user and hence does not correspond to an artificially constructed or artificially rendered object, as is the case e.g. in augmented reality. In other words, the processor may at least partially take over the corresponding identification or retrieval of (actually present) visual information, which may become increasingly intricate, if not impossible, for a patient to achieve with e.g. an increasing number of objects being present. The performance of such task by the processor may hence significantly facilitate the identification of the object of interest and the focusing said object by the patient. Such detection, however, may also be performed, in case a single or only few objects are present, wherein the detection may e.g. confirm the presence or identification of said object. The detection of the object may e.g. be based at least in part on remaining peripheral vision or remaining central vision, but may likewise be provided in case of full loss of peripheral and/or central vision of the patient.

Furthermore, the outputting of the signal for centering the portion of the object of interest relative to the predefined retinal area may facilitate proper perception of the object of interest at the predefined retinal area. In other words, the signal for centering may facilitate that the object, i.e. depending e.g. on the captured image and/or the size thereof, either a portion thereof or in its entirety, is being brought within the corresponding field of view of the patient and to align the corresponding light beam with the predefined retinal area. Such alignment may be enabled by the user or patient accordingly moving his/her head or by translation of the image, as will be described further below.

The user input may be a single user input. In particular, the user input may be a non-visual input, since the object of interest is typically not unambiguously detectable and/or identifiable by the user by vision. For example, the first user input device may comprise or be formed as an audio or voice input device, wherein the patient or user may directly name the object of interest and the processor subsequently verifies whether such object is present by the captured image. Since the patient or, more generally user, as used synonymously herein, is typically interested in a particular object of interest to be detected, said object may be directly input to the processor using the first user input device, for example, using a voice command. The processor thereafter verifies whether said object is actually represented by the captured image.

Alternatively, the processor may e.g. also detect an object being closest to an optical axis of the camera, such that an object-typically corresponding to a gaze direction-may be detected. After having received the user input and having detected the object of interest, the signal for centering the portion of the object of interest is output, thereby facilitating proper positioning of the predefined retinal area relative to the object of interest.

The captured image may be a single image obtained at a time-point corresponding to the received user input or may be a series of images. For example, the user may move his head after having provided the user input or while providing the user input, wherein the processor is advantageously configured to detect the object of interest in the corresponding enlarged overall field of view of the camera.

The system is to be understood as a portable system and, in particular, may comprise a spectacles frame and/or comprise a headband and/or ear stoppers to hold the camera and the projector device.

The processor may be foreseen on the support device or frame, e.g. in an integrated manner with the camera and/or projector device. However, the processor may also be provided separately as a portable device, which is communicatively coupled to the camera and the projector device. Thereby, the weight of the components on the support device or frame may be reduced. The processor may be part of or formed by a control unit of the system and/or may at least partially be established in the form of a control logic forming an interface between the camera, the user input device, and the projector device. For example, the processor may be present in the form of a microcontroller with an input and output interface or corresponding circuitry.

The camera is preferably integrated in a spectacles frame or other wearable device aligning the camera with the field of view or the main viewing direction of the user. In contrast to remote or external cameras, the camera may hence be arranged so as to directly detect the surroundings in front of the user. Accordingly, the processor preferably verifies whether, based on the user input, an object of interest, in particular, as described above, a physically and/or graphically actually present object, is present in the current field of view of the camera, which is hence typically in front of the user. The processor hence preferably does not perform a detection of any objects that may be artificially rendered into the outputted signal, as is the case in e.g. augmented reality.

The camera is preferably positioned in front of the projector device, wherein a main axis of the camera may be aligned with a main axis of the light beam, preferably concentrically. Preferably, the camera and the projector device are arranged in line facing opposite sides of the support device or frame. Thereby, it can be ensured that, when a person equipped with the projector device is looking straight ahead, the person may see exactly what is straight in front of him. In other words, it may hence be easier for the person to focus on a point of interest and, moreover, to allow him to find appropriate orientation in space.

According to a preferred embodiment, the “predefined retinal area” comprises the modified retina area, i.e. an area comprising a retinal implant or a reactivated retinal area.

The predefined retinal area preferably corresponds to a position and size of a modified retinal area, i.e. a retinal implant or a reactivated retinal area. In addition, a native portion of the retina of the patient may remain functionally intact but may not always be aligned with the object of interest. Under such circumstances, the predefined retinal area may correspond to said portion of the retina, such that the portion of the captured image being projected is perceived by said portion of the retina, e.g. with improved optical quality.

Preferably, the processor is configured to detect one or more objects in the captured image based on a first user input and to select the object of interest based on a second user input. As described above, a single user input may suffice, for example, if a limited number of objects are present within the field of view of the camera, i.e. within the captured image. However, if a larger number of objects are present and/or a user is not yet aware of the object of interest, the object of interest is preferably selected in a subsequent step, such that the selection of the object of interest and the corresponding signal(s) output by the processor may be based on a more specific user decision.

In this regard, the system is preferably configured to provide a feedback signal corresponding to the detected one or more objects to the user based on the first user input. For example, a user may initially not be (fully) aware of the objects being present, e.g. in the case of significantly impaired or full loss of peripheral and/or central vision or may have no particular object of interest. The first user input may accordingly result in the detection of one or more objects that are present and corresponding information is provided to the user by means of the feedback signal. Based on the detected objects reported to the user by means of the feedback signal, the user may then provide a second user input selecting the object of interest, such that the processor provides the signal for centering corresponding to the selected object of interest.

The selection by a second step, i.e. the above two-step approach may hence increase specificity of the object of interest and/or relevance of the object of interest to the user. In other words, the user may first retrieve information regarding the object(s) being present and may thereafter decide, which object of interest is to be selected.

The detection of the one or more objects may be based on one or more object detection algorithms. The objects to be detected may e.g. be specific objects based on one or more object categories and/or may depend on their distance relative to a central field of view of the camera. The objects may also be user-defined, e.g. be based on user preferences. By the same token, the objects to be detected may be based on machine learning algorithms using previous object detection data, preferably comprising user-specific object detection data and/or based on user preferences. Such user preferences may be stored in the system, e.g. in a non-volatile storage medium communicatively coupled to the processor.

In addition to the object detection, the processor may perform semantic segmentation and analysis, e.g. to provide a relative position of the one or more objects to each other and/or of portions of a single objection. Thereby, it may e.g. be determined that an object, in particular an object of interest, is arranged adjacent to or on top of another object, e.g. a book is positioned on top of a table. By the same token, it may be determined that a human being is seated on a chair. Accordingly, the semantic segmentation may ensure a more accurate detection of the object of interest.

After having received the first user input, information regarding the objects detected by the processor may be provided in a predefined sequence. For example, the objects indicated in the feedback signal may be provided in an order starting with the object comprising a highest matching score with one or more user preferences. Alternatively, or in addition, objects comprising textual and/or graphical information, e.g. magazines, books, newspapers, or portions thereof, may be given more significance for determining the order of objects in the feedback signal. By the same token, the presence of human beings and/or animals may be of particular interest to the user. The order of the one or more objects is preferably based on the received first user input, which may e.g. correspond to a user prompting whether a particular object or object category is present.

To provide a more natural and/or intuitive perception for the user, feedback regarding the detected objects may be provided in an order depending on the proximity of the objects to the central field of view of the camera and/or in a clockwise or counterclockwise manner.

The system may be communicatively couplable to or may comprise an acoustic unit and may be configured to output the feedback signal as an acoustic input signal to the acoustic unit. By means of an acoustic feedback signal, a user may be provided with readily understandable and perceivable information regarding the one or more detected objects. For example, the acoustic input signal may be provided in the form of artificial speech. Such artificial speech may be provided in a predefined order or sequence of detected objects, as described above. Preferably, the feedback signal comprises semantic information, as described above, such that the feedback signal may provide a caption or abstract of at least a portion of the captured image.

Alternatively, the acoustic signal and, in particular, the artificial speech, may also provide a feedback signal directly related to the (first) user input. That approach may be eligible, if e.g. the user input is provided by means of a corresponding voice command or question. For example, a first user input of the user may comprise a question, whether a particular object of interest or a category of objects is present, such that the feedback signal may confirm the presence or absence of such object. Together with the provision of the feedback signal, i.e. in the affirmative, the processor may output the centering signal for a corresponding (single) object of interest. Such centering signal may e.g. be directly provided, if appropriately defined by the user's question, but may alternatively also require confirmation of the user, e.g. by a second user input.

In addition to the first user input device, the system may comprise at least a second user input device being in communication with the processor, wherein the processor is preferably configured to detect the one or more objects in the captured image based on the first user input from the first user input device and to select the object of interest based on the second user input from the second input device.

The provision of a first and second input device may improve the versatility and interaction between the user and the system. In particular, one of the input devices may be configured to enable a voice command or input, e.g., by comprising a microphone and receiver that are in communication with the processor. By enabling a voice input, the processor may be adapted to detect the one or more objects based e.g. on a specific question of the user and may accordingly provide an optional feedback signal, e.g. as a primary or initial output to indicate to the user, which objects are present. By the same token, a voice input may enable a specific selection of an object of interest or confirmation thereof.

The other at least one input device may furthermore comprise one or more push-buttons. Thereby, a haptic or mechanical input is enabled. Such an approach may reduce e.g. an overall voice input to be applied by the user. It may be advantageous, both, in terms of privacy of the user and to reduce computational effort and potentially expedite the user input. For example, a first user input may be provided by pressing a push button, thereby directly triggering detection of (particular) objects present in the vicinity of the user. Once one or more objects have been detected, the user may select an object of interest e.g. by providing a corresponding voice input, preferably after the outputting of a corresponding feedback signal by the system.

Any such push button, however, may also be used to select an object of interest. A direct selection may e.g. be performed during the outputting of a corresponding feedback signal by the system. To facilitate such selection, irrespective of a current feedback signal, multiple push buttons may be present rather than a single push button. Each push button may preferably correspond to a dedicated object or object category.

The first and second user input device may also be compatible for both the first user input and the second user input. Thereby, the user may trigger the corresponding detection and selection e.g. in a manner that may be adapted to the current environment of the user. The first user input device and the second user input device may also be formed as respective units that are combined within a single user input device. As described above, the user input is particularly a non-visual input, since the object of interest is typically not unambiguously detectable and/or identifiable by the user by vision.

According to a preferred embodiment, the first user input device is formed as a hand-held pointing device. By means of the pointing device, an area of interest for detecting the one or more objects may be defined by a user, once the pointing device has been detected by the camera in the captured image. The pointing device may furthermore comprise one or more push buttons, which may trigger the corresponding detection of the one or more objects by the processor. As described above, respective push buttons may be dedicated to corresponding object categories. Thereby, the pointing towards an area and the pressing of the respective push button may trigger a more specific detection, e.g. without requiring any voice input anymore.

The area of interest may e.g. be adjacent to a predefined end surface of the pointing device. In particular, the area of interest may be directly above or around the predefined end surface in the captured image. The processor may be adapted to identify the predefined end surface by means of a size, a shape, a texture, a material or tissue type, a level of kurtosis, one or more principal axes, and/or a color of the end surface. For example, the pointing device may have e.g. a pen shape or is a pen, marker, or pencil. Under such circumstances, the pointing device may comprise straight edges or contours and may comprise a tip having a particular curvature and/or color. The identification of the tip may optionally be facilitated by the identification of a main longitudinal axis and/or a texture of the tip in the image.

In order to identify the predefined end surface, the processor may use one or more algorithms that use image processing and detects one or more of the above characteristics, e.g. by using one or more feature recognition algorithms. Subsequently, the predefined end surface may be identified using e.g. machine learning or neural networks and/or may use a look-up table or database to match one or more of the identified characteristics with known values or ranges for said characteristics.

The pointing device may also correspond to a dedicated object, preferably in the form of a digital pencil. Advantageously, the processor is adapted to be communicatively coupled to the pointing device and to determine the area of interest and/or the object in the image based on a received orientation information of the pointing device. The pointing device, e.g. the digital pencil, may be equipped with one or more sensors, e.g. (pressure) sensors indicative of the orientation and or interaction with an object of interest, such as a hand-held document. The orientation determined thereby advantageously enables a more precise identification of the predefined end surface and advantageously enables the processor to be adapted to adjust the positioning of the area of interest based on the received orientation so as to improve the identification of the object of interest.

Alternatively, one or more fingers of the user may be used, wherein the predefined end surface is preferably a fingertip. While a communicative coupling is not established thereby, the processor may still be thereby allowed to obtain sufficient information to optionally determine an orientation of the finger. For example, a particular finger, e.g. an index finger, may be identified, wherein a relative angle or distance to one or more adjacent fingers or to the adjacent wrist may enable a calculation or an estimate of the orientation of the index finger and fingertip. Such estimate or calculation may e.g. be based on historical data or on a database with corresponding entries and known orientations or angles of fingers and fingertips. Furthermore, the estimate or calculation may be iteratively improved, e.g. based on machine-learning and a corresponding algorithm.

The predefined end surface may furthermore be uniquely identifiable. As described above, the end surface may correspond to a tip of a dedicated object such as a pen or digital pencil, but may also correspond to a fingertip, e.g. of the right or left index finger of the patient. The unique identification ensures that no inadvertent information is forwarded to the user. Avoidance of provision of inadvertent information may be particularly advantageous, whenever another object, which is not intended for detection of the object of interest, or another finger, which may be present e.g. to stabilize or hold a document, is within the field of view of the camera. In case of e.g. a fingertip, further features such as biometric characteristics may be retrieved from the captured image.

The pointing device may also facilitate the selection of the object of interest. For example, a predefined movement of the pointing device perceived by the camera and evaluated by the processor may confirm a detection area and a near simultaneous selection of an object being present in said area of interest. Such movement may e.g. correspond to a circular or circular movement. Such perceived movement may also initiate an iterative detection and selection, for example, by iteratively specifying the area of interest, potentially being successively narrower. The movement may also correspond to a swiping movement, wherein a predefined swiping direction may confirm a selection of an object of interest, preferably based on an outputted feedback signal.

Since the resolution of the object of interest may depend on the magnification factor of the camera, the user may be caused to request or to rely on an adjustment of the zoom factor so as to be able to identify the object of interest. When the object of interest comprises graphical or text information, the centering signal may e.g. facilitate that the user distinguishes the object of interest from the surroundings, yet an inappropriate zoom factor, i.e. being too high or too low, may result in a resolution of the information, which does not enable the user to identify the presented information. In other words, if the object of interest comprises e.g. text information, an insufficient zoom factor may render the text characters too small to be read, whereas a larger than appropriate zoom factor may result e.g. in the text characters extending beyond the predefined retinal area.

Accordingly, the signal for centering preferably comprises a zoom adjustment signal for the camera. By means of the zoom adjust signal, a magnification factor or focal length of the camera may be adjusted so as to change the angle of view and the breadth of the scene being captured. Thereby, an appropriate resolution may be provided in a semi-automated manner. By providing the correct resolution, the occurrence or likelihood of a crowding effect may be significantly reduced. Furthermore, the zoom adjustment signal may be provided together with a focusing signal for the camera, which may be based on a determined distance of the object of interest relative to the camera. The focusing signal may accordingly adjust the focal plane relative the camera sensor so as to provide a required sharpness of the object of interest. The zoom adjustment signal may also be provided or input successively to the signal for centering, such that the zoom adjustment is preferably performed, once the object of interest has been aligned with the predefined retinal area.

By a preferred embodiment, the signal for centering may be output as a support signal to the user indicating a relative position between the camera and the object of interest and/or a required zoom level of the camera. The object of interest may e.g. not be within the center of the view of the user and/or (at least partially) not be perceived by the predefined retinal area. By means of the support signal, the user, who is provided with the support signal, may hence be guided towards the object of interest, e.g. by corresponding head and/or eye movement, such that the predefined retinal area is brought into alignment with the object of interest. Such guidance in the form of a support signal may facilitate the natural viewing or seeing behavior of a user. Thereby, the user may be required to move his head in order to focus on the object of interest. The system is hence preferably adapted to indicate that the object of interest is not centered and an alignment with the optical axis should be envisaged.

The relative position of the camera and the object of interest may e.g. be determined based on data received from a gyroscope, a magnetometer, an accelerometer, and/or an inertial measurement unit coupled to the camera or system and/or may be based on an eye movement tracking of the user. By a straight-forward approach, a central gaze direction of the user may be assumed, wherein the relative position is determined based on a distance of the object of interest to an optical axis of the camera or center point of the captured image.

The support signal may comprise an acoustic signal, wherein the system preferably comprises and/or is communicatively couplable to an acoustic unit for outputting the acoustic signal. Accordingly, the system may provide guidance by means of voice support, indicating the direction of movement required to center the object of interest on the predefined retinal area. If the acoustic unit or an acoustic module is not integrated in the system, the communicative coupling may be established via an interface of the processor, e.g. in the form of a wireless module, transceiver, or receiver.

The acoustic support signal may e.g. include voice guidance or orientation directions such as “left”, “right”, “up”, and “down”. Furthermore, as described above, a zoom adjustment signal may be provided, resulting in a semi-automated adjustment of the appropriate magnification to identify the object of interest. Alternative, the zoom signal may also be forwarded to the user in the form of an acoustic guidance, e.g. “zoom in” or “zoom out”, thereby ensuring a large degree of control and freedom regarding the desired magnification for the user. Accordingly, the zoom factor may be adjusted by the user, wherein a more subjective perception may be taken into consideration.

Alternatively, or in addition, the system may also comprise and/or is communicatively couplable to one or more portable electromechanical transducers. Thereby, the system may be configured to actuate the one or more portable electromechanical transducers based on the outputted support signal. The support signal may e.g. correspond to one or more vibrations perceivable by the user. Preferably, a direction of movement of the head of the user for centering the object of interest in the field of view of the camera and/or align the object of interest with the predefined retinal area may thereby be established.

The electromechanical transducers are preferably arranged in a wearable item. Such wearable item is preferably configured to extend over the shoulders along the neck and throat area. For example, the wearable item may be formed as a vest or harness. The upper thoracic and cervical anatomical regions have been found to be particularly sensitive for haptic feedback and cause neural stimulations associated with the visual cortex. Any such haptic feedback loop may hence facilitate a normal viewing behavior of the user.

The portable electromechanical transducers may also be arranged in other user portable items, such as a pen and/or within a spectacle frame. Thereby, multiple functions of the system may be combined in a single device without requiring additional separate items for providing haptic feedback.

The support signal may also be established in the form of visually perceivable information. Accordingly, the processor may be configured to forward the support signal to the projector device, wherein the support signal preferably establishes a predefined pattern for a portion of the light beam not corresponding to the object of interest. Thereby, the predefined pattern may comprise one or more guiding lines originating from the object of interest and indicating a direction for centering the camera and/or the predefined retinal area.

Such guiding lines may correspond to a perceivable yet not disturbing light intensity that is preferably in contrast with the remaining portion of the captured image being output to the user. The guiding lines may each be connected with a different outer position of the object of interest and form a respective angle representative for an offset to the camera's central pointing direction and/or predefined retinal area. The one or more guiding lines may be continuously adapted based on adjustments of the camera position or of the position of the predefined or modified retinal area to facilitate a correct positioning and alignment.

Instead of lines, other guiding patterns may also be provided. For example, alternatively, or in addition, a perceivable intensity gradient may be applied, wherein the highest light intensity may correspond to or be adjacent to the object of interest.

For the portion of the captured image which does not correspond to the object of interest, the signal output to the projector device may define a predefined and/or standardized pattern. Preferably, in comparison with the object of interest, the predefined and/or standardized pattern may be a pattern with reduced brightness, a blurred pattern, a pattern with reduced resolution, an essentially homogeneous pattern, and/or an essentially continuous pattern.

Such predefined and/or standardized patterns ensure that the user is enabled to continuously perceive the same size of the light beam and corresponding visual information. Thereby, dysfunction of the system as a whole may be prevented. Furthermore, in case of a misalignment of the predefined or modified retinal area, the user may more easily align the predefined or modified retinal area with the position of the light beam, since the size of the light beam is not reduced. Accordingly, only minor eye movements may be required to adjust the position of the eye to the predefined (natural seeing) position.

The visual information to be detected by the camera may significantly go beyond what is perceived by the user as a result of the light beam and the corresponding predefined or modified retinal area. Visual information loss may be due to the size of the predefined or modified retinal area on the one hand and the amount of information that may be processed or transmitted in subsequent stimulation of the corresponding nerve end. For example, a retinal implant may be characterized by a limited resolution of between e.g. 400 and 2500 pixels.

Thus, the following typically holds: Even in case of proper alignment of the camera and the predefined or modified retinal area, the user may have difficulties to identify the context and interpret the object of interest. For example, when the user starts reading a document (such as a book or newspaper), objects surrounding the text (such as images, bright areas, and/or patterns that are out of focus) may render it more difficult to identify the relevant information.

Hence, to facilitate identification of the relevant information, the signal output to the projector device may essentially only define the object of interest.

Thereby, essentially only the object of interest within the predefined region may be perceived by the user and other, not relevant information, may be sorted out or discarded. To further increase the perceivability of the object of interest or portion thereof, the object of interest may, alternatively or in addition, be converted so as to provide an abstract and/or simplified representation thereof. That approach may, be put into practice by e.g. modifying the brightness and/or contrast and/or by outputting the pattern of interest in a standardized manner, e.g. a predefined text or image layout. As described above for the portion of the captured image which does not correspond to the object of interest, the signal output to the projector device and corresponding to the object of interest may hence also define a predefined and/or standardized pattern corresponding to or adapted to the object of interest.

The portion of the captured image adjacent the object of interest may also be combined with a standardized and/or predefined pattern, which may be advantageous e.g. to facilitate a reading flow upon movement of the camera.

It is to be understood that the support signal may be provided in a combinatory manner, such that e.g. an acoustic signal may be output along with an electromechanical or haptic signal and/or a visual guidance. In other words, the support signal may be output to multiple actuators so as to effect respective support or guidance signals. Such multi-sensory integration may be particularly advantageous for users or patients with impaired vision, wherein e.g. audio-tactile maps may be adapted to the respective user.

In an alternative preferred embodiment, the processor may be configured to output a signal corresponding to a portion of the captured image comprising the object of interest to the projector device and to output the signal for centering said object of interest relative to the predefined retinal area to the projector device.

By outputting the portion of the captured image with the object of interest and outputting the centering signal to the projector device, the object of interest may hence be brought in alignment with the predefined retinal area. Thereby, the object of interest is perceived at a central position. In other words, the processor may be configured to translate the object of interest by outputting the light beam with the object of interest at a central position relative to predefined retinal area and/or a central optical axis of the camera. Rather than providing a user guidance by means of a support signal, the system is thereby configured to realize a centering of the object in an automated manner. An offset of the object of interest may be taken into account by a corresponding translation using the signal for centering the object of interest. Accordingly, the signal for centering may adjust the x-and/or y-coordinates of the object of interest as captured in the image so as to provide a central positioning of the object of interest and/or alignment with the predefined retinal area.

Preferably, the processor is configured to include a zoom adjustment and autofocus, such that the object of interest is presented to the user in a manner being optimized for identification. Alternatively, or in addition, the processor may also output a zoom adjustment signal and/or focus adjustment signal to the camera. As a result, the object of interest may be captured with improved resolution, preferably prior to outputting the signal to the projector device.

While potentially realizing a less natural viewing experience, the automated approach may be particularly advantageous, whenever a large number of objects is present. Such a larger number of objects may not be readily distinguishable from each other or would thus significantly impair proper centering and zooming by a user. Furthermore, automated centering is not dependent on further components of the system, such that the system may be based on a more compact design. The automated centering may also be established in an alternating mode to a supported or guided centering.

Preferably, the processor is configured to output a signal corresponding to a portion of the captured image based on a received first user input. The portion of the captured image may be in a central field of view of the camera at a time point of the received first user input. The processor may be configured to output the signal corresponding to a portion of the captured image comprising the object of interest and the signal for centering said object of interest relative to the predefined retinal area based on a subsequently received second user input.

In other words, the user may effect a first user input so as to define the relevant area for the one or more objects to be detected. Thereby, said area preferably corresponds to the image captured at the time point of providing the first user input. That approach may result in a still or “freeze” image for the processor to be processed. Such an approach may be particularly advantageous in the presence of a large number of objects and/or in case of a large field of view of the camera, which may render it difficult to detect the presence of (particular) objects in a real-time manner. The provision of the still image thus ensures that detection is performed on a particular image having been captured.

Furthermore, the provision of an initial still image also reduces a potentially confusing or dazzling effect to a user. Such an effect may e.g. be due to the abundance of information and/or a changing relative position of the predefined retinal area or camera and the object of interest. For example, an object moving within an environment containing a large amount of distinct visual information may imply that the user may get lost when following the object of interest. Provision of the still image, however, ensures that the object may be accurately identified while, at the same time, the user may be assured that detection has been initiated.

Once the object of interest has been identified, the processor may output the signal corresponding to the portion of the captured image comprising the object of interest, as described above.

The processor may be configured to modify the centering signal in a predefined direction and/or predefined speed. Thereby, the portion of the object of interest being presented to the user may be changed. Such an embodiment may be particularly advantageous, if the object of interest comprises graphical information and/or text information. A user may e.g. be interested in a particular article, e.g. in a newspaper, or a chapter in a book. Movement of the object of interest may enable a more natural observation experience or may mimic a reading movement.

For example, a user may perform a first user input by requesting information about whether an article on a particular topic, i.e. comprising one or more reference keywords, e.g. in a respective title, is present at all. By the same token, the user may also point to an article and may request information about whether said article comprises the corresponding content. If such an article is actually identified, the user may select said article. The processor is caused to output the corresponding signal and to modify the centering signal. Scanning of the article that is perceivable by the user, may follow.

In order to provide an improved user control, the processor may be configured to modify the predefined direction and/or the predefined speed based on a received second or third user input. For example, the first user input may directly allow the system to acknowledge the presence of the object of interest and/or may result in a selection of the object of interest. Thereafter, scanning of the object of interest with a predefined direction of movement and/or a predefined speed may follow. The modification may facilitate the full perception of the content of the object of interest, e.g. of an image or text portion and may hence simplify e.g. a scanning movement. In particular, such modification may predefine a modification of the signal for centering from left to right and from top to bottom.

However, selection or initiation of the scanning step may also (additionally or alternatively) be based on a second user input. Accordingly, the adjustment or modification of the predefined direction and/or predefined speed may be based on the second user input or third (or subsequent) user input. The processor may adjust the signal for centering so as to provide a movement over the object of interest from left to right and from top to bottom, corresponding to a usual reading movement of a user. The second or third user input may e.g. result in a change of the scanning or reading speed.

While the adjustment may be based on a user input performed using e.g. a voice command, the speed of movement or scanning is preferably controlled by one or more push buttons, allowing the user to exert a more direct and/or accurate control.

According to another aspect of the invention, a computer implemented method for projecting at least a portion of an object of interest onto a predefined retinal area of a human eye of a user is suggested, comprising the steps of:
receiving a user input from a first user input device;
capturing an image using a camera; and
outputting a signal corresponding to a portion of the captured image to a projector device.

According to the invention, an object of interest in the captured image is detected based on the user input. A signal for centering said object of interest relative to the predefined retinal area is provided as output.

The computer implemented method may e.g. be stored or embodied on a non-volatile computer readable storage medium, e.g. in the form of executable instructions. A processor may e. g. execute the corresponding operations. In particular, the computer implemented method may be provided as a computer program product embodied on such computer readable storage medium. The features and advantages discussed with respect to the system also apply to the method and vice versa.

In accordance with the above, it is to be understood that the “modified retinal area” as used herein may both relate to a retinal area of the human eye that has been modified to restore photosensitive behavior through implantation of a retinal prosthesis and to modification by “optogenetics”. The “modified retinal” area preferably corresponds to or constitutes the “predefined retinal” area. However, the predefined retinal area may also constitute a native portion of the retina of the patient that remains functionally intact, but may not always be aligned with the object of interest. Under such circumstances, the predefined retinal area may correspond to said portion of the retina, such that the part of the object of interest being projected is perceived by said portion of the retina, e.g. with improved optical quality.

### BRIEF DESCRIPTION OF THE DRAWINGS

The present invention will be more readily appreciated by reference to the following detailed description when being considered in connection with the accompanying drawings in which:

FIG. 1 shows a schematic depiction of a system according to the invention and its interrelationship with its surroundings and a predefined or modified retinal area;

FIG. 2 shows a schematic depiction of distinct steps for detecting and selecting an object of interest for establishing a support signal for centering the object of interest;

FIG. 3 shows a schematic depiction of different steps for detecting and selecting an object of interest for centering the object of interest by translation; and

FIG. 4 schematically shows detection of objects in a field of view and a corresponding provision of a feedback signal.

### DETAILED DESCRIPTION OF PREFERRED EMBODIMENTS

In the following, the invention will be described in more detail with reference to the accompanying figures. In the Figures, like elements are denoted by identical reference numerals and repeated description thereof may be omitted in order to avoid redundancies.

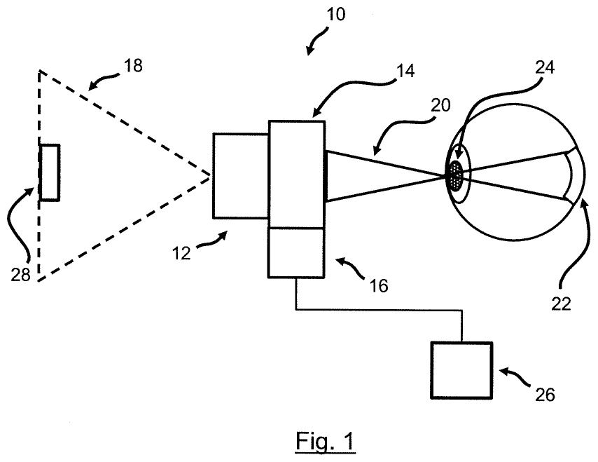

In FIG. 1, a schematic depiction of a system 10 according to the invention is shown. The system 10 comprises a camera 12 and a projector device 14, which are coupled to each other via a processor 16. The processor 16 is shown as being in close arrangement with the other components of the system 10 and may e.g. be attached to or integrated in the projector device 14. Alternatively, however, the processor 16 may also be realized as a separate component so as to distribute the overall weight of the system 10 on distinct components. This may facilitate the portability of the system 10, since the camera 12 and projector device 14 are preferably attached to a head-worn frame, such as a spectacles frame. Additional weight gain may be inconvenient for a user or patient. In such an arrangement of separate components, e.g. carried in a pocket or by a body-worn strap, communication between the camera 12, the processor 16, and the projector device 14 may be ensured by means of corresponding wiring or by a wireless technology, e.g. in a wireless configuration.

In operation, one or more images or a series of images may be captured by the camera 12. The image is preferably based on the field of view 18 of the camera 12 and a set focus of the camera 12 and, potentially, also based on a focal distance and/or aperture of the camera. In the image, an object of interest 28 (such as written text information or other visual content that may be of interest to the user or patient) is present. The user may not be (fully) aware of the object of interest 28, for example, if the object of interest 28 is present at an offset to a central optical axis of the camera 12 such that it is not visually perceivable by the user.

In order to determine whether said object of interest 28 is present, the user is provided with a user input device 26, which is communicatively coupled with the processor 16. Based on a user input performed by the user input device 26, the processor 16 may receive an image or sequence of images captured by the camera 12. The processor 16 preferably receives the image(s) as an input signal. Based on the input signal from the camera 12 and the user input from the user input device 26, the processor 16 may detect whether one or more objects are present and/or may determine whether the object of interest 28 is present.

If the object of interest 28 is present and the processor 16 is provided with a corresponding selection command, the processor 16 may output a signal corresponding to a portion of the captured image to the projector device 14 and may output a signal for centering said object of interest 28 relative to the predefined retinal area 22. In other words, the signal outputted by the processor 16 forms an input signal for the projector device 14. The projector device 14 is typically adapted to project a light beam 20, preferably a pulsed light beam, into a human eye based on an inputted signal.

The arrangement of the projector device 14 and the size of the light beam 20 are chosen such that the light beam 20 enters the human eye via the pupil 24. Preferably, the entire light beam 20 may be provided through the pupil 24. After entry and passing through the eye lens, the light beam 20 progresses towards the retina and is projected to the predefined retinal area 22. In the present non-limiting example, the predefined retinal area 22 is a modified retinal area. According to the present embodiment, the modified retinal area comprises a retina implant.

The system 10 thereby enables that visual information obtained by the camera 12 may be processed by the processor 16 and may be forwarded to the user by means of the corresponding light beam 20 projected by the projector device 14. At the level of the retina implant, the received light, preferably pulsed infrared light, is converted into signals (current pulses). These pulses stimulate adjacent tissue, cells or nerve end of the user, in particular retinal cells. The user may perceive the processed visual information, at least in part, which may be considered as a modified retinal area image.

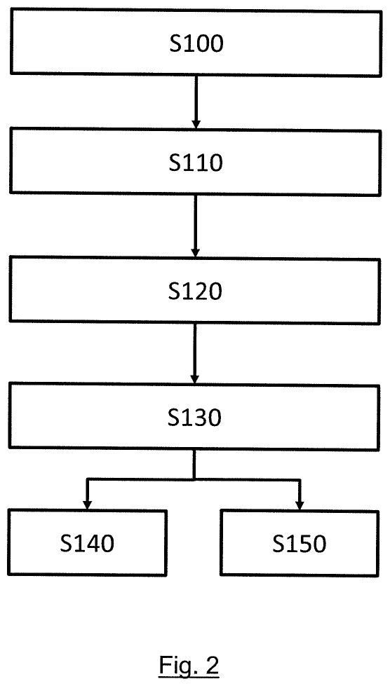

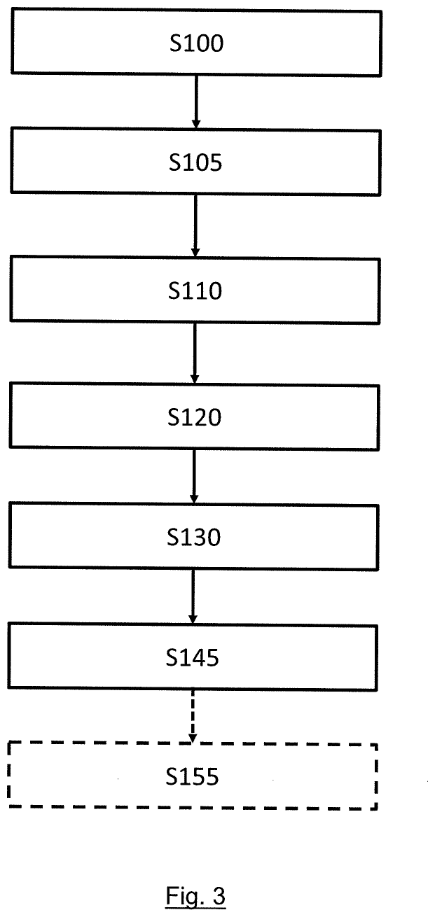

The signal for centering the object of interest may furthermore facilitate that the user perceives the object of interest 28 at the predefined retinal area 22, as shown in further detail by the embodiments depicted in FIGS. 2 and 3.

Accordingly, FIG. 2 shows a schematic depiction of different steps for detecting and selecting an object of interest 28 for providing a support signal for centering the object of interest 28.

In a first step, S100, a user input is provided by using an input device. The user input indicates to the processor that one or more objects or a particular object of interest are to be detected. Accordingly, an image is captured using the camera, which is provided as an input to the processor. Based on the provided user input in step S100, the processor detects one or more objects in the captured image in step S110, e.g. by using feature recognition algorithms. The user is informed about the detected one or more objects by means of a feedback signal that is outputted in step S120 by the system, e.g. via an acoustic interface and/or a tactile interface.

The user may then select an object of interest in step S130. The feedback signal may e.g. confirm the presence of a particular object of interest that has been queried by means of the first user input. Hereby, the feedback signal may simply confirm the presence and may result in the selection of said object of interest or the user may be required to actively confirm the selection by means of a corresponding input in step S130. Alternatively, the feedback signal may comprise information regarding some or a plurality of objects being present in the captured image. Thereafter, the user is required to perform a selection of one of the objects as an “object of interest”.

After the object of interest has been selected, the processor outputs a signal to the projector device corresponding to a portion of the captured image, step S140. Such a signal may also be generated during the previous steps S110, S120, and S130, such that the user is continuously supplied with visual information. After the object of interest has been selected in step S130, a signal is output for centering the object of interest in step S150. According to the present, non-limiting example, said centering signal may correspond to audio signals or haptic signals or combinations thereof, which may be output using an acoustic module or speaker and one or more electromechanical transducers, respectively. Thereby, the user is guided towards the object of interest. The natural viewing behavior of the user is thus facilitated or supported.

In the embodiment according to FIG. 3, a semi-automated centering of the object of interest is provided. As described above, such an approach may be particularly advantageous in the presence of a larger number of objects and/or in case of a larger field of view of the camera. Such conditions may render it difficult to detect the presence of (particular) objects in a real-time manner. Accordingly, rather than continuously providing a signal corresponding to a portion of the captured image to the projector device, a still image may be generated in step S105. That step ensures that the detection may be performed on a particular image having been captured. Furthermore, such a step may significantly contribute to avoid confusion, when the object of interest is translated to a central position relative to the predefined retinal area.

After the object of interest has been selected in step S130, a signal is output to the projector device corresponding to a portion of the captured image comprising the object of interest. Said portion with the object of interest is translated so as to be arranged at a central viewing position relative to the predefined retinal area, as indicated in step S145.

In a preferred embodiment, the object of interest may correspond to text information in the form of an article. Once the article has been detected in step S120 and has been selected in step S130, the processor may be configured to modify the centering signal in a predefined direction and/or predefined speed. Thereby, the portion of the object of interest being presented to the user may vary. Accordingly, a scanning movement may be performed, which facilitates reading the article to the user by mimicking a reading movement.

In order to ensure an improved user control, the processor may optionally be configured to modify the predefined direction and/or the predefined speed based on a received adjustment signal for centering the object of interest in step S155. For example, the user may trigger a further user input, which results in a corresponding change in the speed of the centering signal, i.e. change of the scanning of the article.

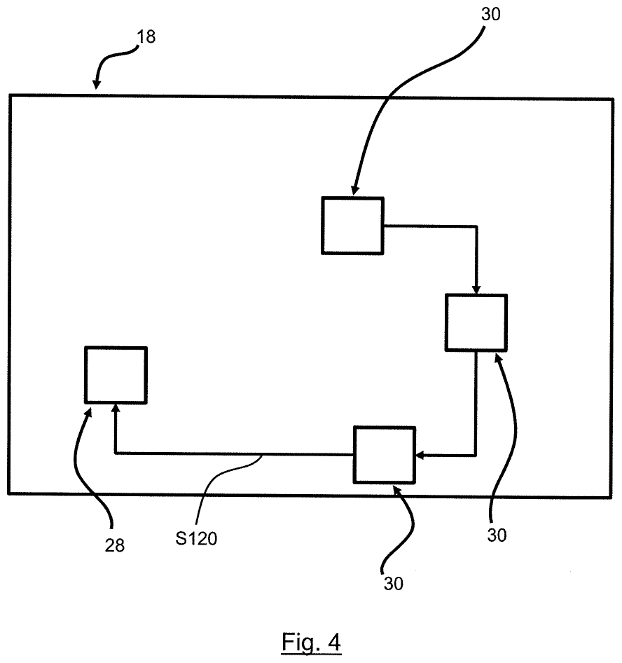

In FIG. 4, an example is schematically depicted. Objects in a field of view 18 of the camera are detected and a corresponding feedback signal is provided.

As shown, a non-limiting number of four objects 30 is depicted. They are present within the field of view 18 of the camera and may be detected by the processor in the corresponding captured image. One of the objects 30 corresponds to an object of interest 28, which may be defined based on a second user input in the form of a particular selection among the objects 30. In the present example, the processor outputs a feedback signal by step S120 in an acoustic form, e.g. artificial voice or speech, indicating information of the objects 30, which allow the user to identify the respective objects or at least identify their relevance. The feedback signal of step S120 is output for the objects in a predefined order. It may start with the object 30 being closest to the central axis or center point of the captured image and may continue with the other objects in e.g. a clockwise (or e.g. counter-clockwise) manner. Preferably, their offset to the center point is also taken into consideration.

According to the example, the object of interest 28 is positioned at the end of the clockwise rotation and is furthermore positioned with the largest offset to the center point of the captured image. However, it is understood that a different order may be chosen, e.g. based on user preferences and/or previous detection results and user feedback regarding their relevance. If a specific detection request is made for a particular object of interest by means of the initial user input, the feedback signal may also be generated for the object 30 matching or having a highest matching score for the criteria set for the object of interest.

It will be obvious for a person skilled in the art that these embodiments and items only depict examples of a plurality of possibilities. Hence, the embodiments shown here should not be understood to form a limitation of these features and configurations. Any possible combination and configuration of the described features can be chosen according to the scope of the invention.

### LIST OF REFERENCE NUMERALS

10 System
12 Camera
14 Projector device
16 Processor
18 Camera field of view
20 Light beam
22 Modified retinal area
24 Eye pupil
26 User input device
28 Object of interest
30 Object
S100 Provide user input
S105 Generate still image
S110 Detect object(s)
S120 Provide feedback signal
S130 Perform object selection
S140 Output signal of a portion of a captured image
S150 Output signal for centering object of interest
S145 Output signal of a portion of a captured image with centered object of interest
S155 Adjust signal for centering object of interest

1. A system for projecting at least a portion of an object of interest onto a predefined retinal area of a human eye of a user, comprising:
a camera for capturing an image,
a projector device for projecting a light beam into a human eye based on an inputted signal,
at least a first user input device, and
a processor being in communication with the camera, at least the first user input device and the projector device,
wherein the processor is configured to detect at least a portion of the object of interest in a captured image based on a user input received from the first user input device and to output a signal corresponding to a portion of the captured image to the projector device and to output a signal for centering at least said portion of the object of interest relative to the predefined retinal area.

2. The system according to claim 1, wherein the processor is configured to detect one or more objects in the captured image based on the first user input and to select the object of interest based on a second user input.

3. The system according to claim 2, wherein the system is configured to provide a feedback signal corresponding to the detected one or more objects to the user based on the first user input.

4. The system according to claim 3, wherein the system is communicatively couplable to or comprises an acoustic unit and to output the feedback signal as an acoustic input signal to the acoustic unit.

5. The system according to claim 2, further comprising at least a second user input device being in communication with the processor, wherein the processor is configured to detect the one or more objects in the captured image based on the first user input from the first user input device and to select the object of interest based on the second user input from the second input device.

6. The system according to claim 1, wherein the first user input device is formed as a hand-held pointing device.

7. The system according to claim 1, wherein the signal for centering comprises a zoom adjustment signal for the camera.

8. The system according to claim 1, wherein the signal for centering is output as a support signal to the user indicating a relative position between the camera and the object of interest and/or a required zoom level of the camera.

9. The system according to claim 8, wherein the support signal comprises an acoustic signal, wherein the system preferably comprises and/or is communicatively couplable to an acoustic unit for outputting the acoustic signal.

10. The system according to claim 8, wherein the system comprises and/or is communicatively couplable to one or more portable electromechanical transducers and wherein the system is configured to actuate the one or more portable electromechanical transducers based on the outputted support signal.

11. The system according to claim 8, wherein the processor is configured to provide the support signal to the projector device, the support signal defining a predefined pattern for a portion of the light beam not corresponding to the object of interest, the predefined pattern comprising one or more guidelines originating from the object of interest and indicating a direction for centering the camera and/or the predefined retinal area.

12. The system according to claim 1, wherein the processor is configured to output a signal corresponding to a portion of the captured image comprising the object of interest to the projector device and to output the signal for centering said object of interest relative to the predefined retinal area to the projector device.

13. The system according to claim 12, wherein the processor is configured to output a signal corresponding to a portion of the captured image based on a received first user input, the portion of the captured image being in a central field of view of the camera at a time point of the received first user input, wherein the processor is configured to output the signal corresponding to a portion of the captured image comprising the object of interest and the signal for centering said object of interest relative to the predefined retinal area based on a subsequently received second user input.

14. The system according to claim 12, wherein the processor is configured to modify the signal for centering in a predefined direction and/or predefined speed.

15. The system according to claim 14, wherein the processor is configured to modify the predefined direction and/or the predefined speed based on a received second or third user input.

16. A computer implemented method for projecting at least a portion of an object of interest onto a predefined retinal area of a human eye of a user, comprising the steps of:
receiving a user input from a fist user input device;
capturing an image using a camera; and
outputting a signal corresponding to a portion of the captured image to a projector device;
wherein an object of interest in the captured image is detected based on the user input and a signal for centering said object of interest relative to the predefined retinal area is output.
